(19) United States
(12) Patent Application Publication
(10) Pub. No.: US 2005/0192273 A1
(43) Pub. Date: Sep. 1, 2005

(54) Therapy for psychoses combining an atypical antipsychotic and an mglu2/3 receptor agonist

(76) Inventors: Bryan Glenn Johnson, Indianapolis, IN (US); Darryle, Darwin Schoepp, Indianapolis, IN (US)

Correspondence Address: ELI LILLY AND COMPANY;PATENT DIVISION, P.O. BOX 6288, INDIANAPOLIS, IN, 46206-6288, US

(21) Appl. No.: 10/509,772

(22) PCT Filed: Mar. 21, 2003
(86) PCT No.: PCT/US03/07283

§ 371 Date: Sep. 28, 2004

Related U.S. Application Data
(60) Provisional application No. 60/369,771, filed on Apr. 3, 2002.

(51) Int. Cl. A61K 31/5415; A61K 31/195

(52) U.S. Cl. 514/225.8; 514/566; 514/562

(57) ABSTRACT

The present invention provides for a pharmaceutical composition and methods for treating psychosis comprising the combination or a first component which is an atypical antipsychotic with a second component which is a mGlu2/3 receptor agonist. The present invention also provides for a pharmaceutical composition and method of treating a psychiatric disorder comprising the combination of a first component which is an atypical antipsychotic with a second component which is a compound which allosterically enhances receptor activity for mGlu2 and/or mGlu3.

### FIELD OF THE INVENTION

The present invention provides for a pharmaceutical composition and methods for treating a psychiatric disorder comprising the combination of a first component which is an atypical antipsychotic with a second component which is a mGlu2/3 receptor agonist. The present invention also provides for a pharmaceutical composition and method of treating a psychiatric disorder comprising the combination of a first component which is an atypical antipsychotic with a second component which is a compound which allosterically enhances receptor activity for mGlu2 and/or mGlu3.

### BACKGROUND OF THE INVENTION

Psychoses are serious mental illnesses characterized by defective or lost contact with reality. Psychotic patients may also suffer hallucinations and delusions as part of their disease. Psychoses exact a tremendous emotional and economic toll on patients, their families, and society as a whole. While the mechanisms underlying these diverse disease states are poorly understood, recently discovered therapies are offering new hope for the treatment of psychotic patients. Progress in the treatment of psychotic conditions has been achieved through the introduction of new, atypical antipsychotic agents.

While the overall profile of atypical antipsychotics (e.g. clozapine, olanzapine) is superior to that of traditional agents (e.g. haloperidol), these agents still produce significant side-effects (e.g. CNS depression, weight gain, sexual dysfunction) which reduce the patients compliance and ultimately leads to relapses of illness and thus negatively impacts the life-long course of this disease. Also atypicals only minimally reverse many aspects of this illness such as negative symptoms (e.g. mood and affect, cognitive dysfunction). The discovery of new agents which could be used in combination with atypical drugs to enhance their effectiveness at lower doses and/or increase their overall effectiveness against negative symptoms would be a considerable advance in the medical treatment of schizophrenia.

One approach to this problem is to design novel agents that modulate the glutamate systems of the brain, as opposed to atypicals which target monoamine systems (dopamine, serotonin) (so called glutamate hypothesis of schizophrenia). As discussed below, the most accepted test for novel glutamatergic agents involves finding drugs which can reverse the actions of psychotomimetic agents such as phencyclidine (PCP) in animals. Schoepp D. D. and Marek G. J., Current Drug Targets—CNS and Neurological Disorders, 1:215-225 (2002); Moghaddam, B.; Adams, B. W. Science, 281, 1349 (1998).

PCP and PCP-like drugs (e.g. ketamine, MK-801) are non-competitive NMDA receptor antagonists. Anis, N. A.; Berry, S. C.; Burton, N. R., Lodge, D. British Journal of Pharmacology, 1983, 79, 565. The glutamate hypothesis of schizophrenia is supported by the clinical observation that these compounds produce schizophrenia-like symptoms in volunteers and can worsen symptoms in people with schizophrenia. Halberstadt, A. L. Clinical Neuropharmacology, 1995, 18, 237; Krystal, J. H.; Belger, A.; D'Souza, C.; Anand, A.; Chamey, D. S.; Aghajanian, G. K.; Moghaddam, B. Neuropsychopharmacology, 1999, 22, S143. In particular, PCP appears to better model schizophrenia in humans than other agents (such as amphetamine), including producing both positive and negative symptoms. The recognition that other classes of NMDA receptor antagonists such as amino acid competitive antagonists also produced schizophrenia-like effects in humans has further supported the glutamate, or NMDA receptor hypofunction hypothesis of schizophrenia. Rockstroh, S.; Emre, M.; Tarral, A.; Pokomy, R. Psychopharmacology, 1996, 124, 261; Olney, J. W.; Farber, N. B. Arch. Gen. Psychiatiy, 1995, 52, 998; Olney, J. W.; Farber, N. B. Neuropsychopharmacology, 1995, 13, 355. In an attempt to translate this information into a useful animal model, many years of preclinical research on the actions of PCP and PCP-like drugs have been performed. Atypical antipsychotics have been shown to be active in the PCP animal model of schizophrenia, but are not fully effective in this model unless higher doses which produce significant side effects such as CNS depression or motor performance impairment. Cartmell, J.; Monn, J. A.; Schoepp, D. D. Journal of Pharmacology and Experimental Therapeutics, 1999, 291, 161. These new atypical antipsychotic agents, therefore, while holding the promise of improving the lives of psychotic patients immeasurably, may not be sufficient to treat every psychotic patient.

### SUMMARY OF THE INVENTION

The present invention provides a pharmaceutical composition which comprises a first component which is an atypical antipsychotic, and a second component which is a mGlu2/3 receptor agonist.

The invention also provides a method for treating a patient suffering from or susceptible to a psychiatric disorder, comprising administering to said patient an amount of a first component which is an atypical antipsychotic, in combination with an amount of a second component which is a mGlu2/3 receptor agonist.

The present invention also provides a pharmaceutical composition which comprises a first component which is an atypical antipsychotic, and a second component which is an mGlu2 potentiator.

The present invention further provides for a method for treating a patient suffering from or susceptible to a psychiatric disorder, comprising administering to said patient an amount of a first component which is an atypical antipsychotic, in combination with an amount of a second component which is an mGlu2 potentiator.

### BRIEF DESCRIPTION OF DRAWING

FIG. 1 depicts the examination of the combination of a representative atypical antipsychotic, clozapine, and representative second component (1R,4S,5S,6S)-4-[(2′S)-(2′-Amino)-propionyl]amino-(2-sulfonylbicyclo[3.1.0]hexane)-4,6-dicarboxylic acid (LY404039), a mGlu2/3 agonist, for their ability to influence phencyclidine (PCP)-induced motor activations in rats, by using an automated behavioral system.

FIG. 2 depicts the examination of the combination of a representative atypical antipsychotic, clozapine, and a representative second component 1R,4R,5S,6R-4-amino-(2-oxabicyclo[3.1.0]hexane)-4,6-dicarboxylic acid (LY379268), a mGlu2/3 receptor agonist, for their ability to influence phencyclidine (PCP)-induced motor activations in rats, by using an automated behavioral system.

FIG. 3 depicts the examination of the combination of a representative atypical antipsychotic, clozapine, and a representative second component (1S,2R,4S,5S,6S)-2-amino-4-fluorobicyclo[3.1.0]hexane-2,6-dicarboxylic acid (LY459477), a mGlu2/3 receptor agonist, for their ability to influence PCP-induced motor activations in rats, by using an automated behavioral system.

FIG. 4 depicts the examination of the combination of a representative atypical antipsychotic, clozapine, and a representative second component (+)-2-amino bicyclo[3.1.0]hexane-2,6-dicarboxylic acid (LY354740), a mGlu2/3 receptor agonist, for their ability to influence PCP-induced motor activations in rats, by using an automated behavioral system.

FIG. 5 depicts the examination of the combination of a representative atypical antipsychotic, olanzapine, and a representative second component, LY4Q4039, a mGlu2/3 receptor agonist, for their ability to influence PCP-induced motor activations in rats, by using an automated behavioral system.

FIG. 6 depicts the examination of the combination of a representative atypical antipsychotic, olanzapine, and a representative second component, LY379268, a mGlu2/3 receptor agonist, for their ability to influence PCP-induced motor activations in rats, by using an automated behavioral system.

FIG. 7 depicts the examination of the combination of a representative atypical antipsychotic, olanzapine, and a representative second component, LY459477, a mGlu2/3 receptor agonist, for their ability to influence PCP-induced motor activations, by using an automated behavioral system.

FIG. 8 depicts the examination of the combination of a representative atypical antipsychotic, olanzapine, and a representative second component, LY354740, a mGlu2/3 receptor agonist, for their ability to influence PCP-induced motor activations in rats, by using an automated behavioral system.

### DETAILED DESCRIPTION OF THE INVENTION

The Compounds

In the general expressions of the present invention, the first component is a compound which acts as an atypical antipsychotic. The essential feature of an atypical antipsychotic is less acute extra pyramidal symptoms, especially dystonias, associated with therapy as compared to a typical antipsychotic such as haloperidol. Clozapine, the prototypical atypical antipsychotic, differs from the typical antipsychotics with the following characteristics: (1) greater efficacy in the treatment of overall psychopathology in patients with schizophrenia nonresponsive to typical antipsychotics; (2) greater efficacy in the treatment of negative symptoms of schizophrenia; and (3) less frequent and quantitatively smaller increases-in serum prolactin concentrations associated with therapy (Beasley, et al., Neuropsychopharmacology, 14(2), 111-123, (1996)). Clozapine, 8-chloro-1-(4-methyl-1-piperazinyl)-5H-dibenzo[1,4]diazepine, is described in U.S. Pat. No. 3,539,573. Atypical antipsychotics include, but are not limited to:
Olanzapine, 2-methyl-4-(4-methyl-1-piperazinyl)-10H-thieno[2,3-b][1,5]benzodiazepine, is a known compound and is described in U.S. Pat. No. 5,229,382 as being useful for the treatment of schizophrenia, schizophreniform disorder, acute mania, mild anxiety states, and psychosis as described and claimed in U.S. Pat. No. 5,229,382; a polymorph form is disclosed in U.S. Pat. No. 5,736,541;
Risperidone, 3-[2-[4-(6-fluoro-1,2-benzisoxazol-3-yl)piperidino]ethyl]-2-methyl-6,7,8,9-tetrahydro-4H-pyrido[1,2-a]pyrimidin-4-one, and its use in the treatment of psychotic diseases are described in U.S. Pat. No. 4,804,663;
Sertindole, 1-[2-[4-[5-chloro-1-(4-fluorophenyl)-1H-indol-3-yl]-1-piperidinyl]ethyl]imidazolidin-2-one, is described in U.S. Pat. No. 4,710,500. Its use in the treatment of schizophrenia is described in U.S. Pat. Nos. 5,112,838 and 5,238,945. U.S. Pat. Nos. 4,710,500; 5,112,838; and 5,238,945;
Quetiapine, 5-[2-(4-dibenzo[b,f][1,4]thiazepin-11-yl-1-piperazinyl)ethoxy]ethanol, and its activity in assays which demonstrate utility in the treatment of schizophrenia are described in U.S. Pat. No. 4,879,288, Quetiapine is typically administered as its (E)-2-butenedioate (2:1) salt; and
Ziprasidone, 5-[2-[4-(1,2-benzoisothiazol-3-yl)-1-piperazinyl]ethyl]-6-chloro-1,3-dihydro-2H-indol-2-one, is typically administered as the hydrochloride monohydrate.
   The compound is described in U.S. Pat. Nos. 4,831,031 and 5,312,925. Its activity in assays which demonstrate utility in the treatment of schizophrenia are described in U.S. Pat. No. 4,831,031. Additional atypical antipsychotic agents may be discovered beyond those specifically mentioned here. Antipsychotics which may be disclosed in the future may form the first component of the present invention.

Similarly, when the invention is regarded in its broadest sense, the second component compound is a compound which functions as a mGlu2/3 receptor agonist. The measurement of a compound's activity in that utility may be identified for example by using the following experiment.

The affinity of a test compound for metabotropic glutamate receptors may be demonstrated by the selective displacement of [³H]-2S-2-amino-2-(1S,2S-2-carboxycyclopropan-1-yl)-3-(xanth-9-yl)propionic acid ([³H]-LY341495) (17.5 Ci/mmol). The binding of [³H]-LY341495 is conducted with crude membranes from cell lines expressing human mGlu2 and mGlu3 receptors which are derived as described by Johnson B. G., et al., Neuropharmacology, 38: 1519-1529 (1999).

The ability of a test compound to act as an agonist at negatively coupled cAmp-linked metabotropic glutamate receptors may be measured using the following method. Cell lines stably expressing mGlu2, mGlu3 may be derived as previously described in Schoepp D. D. et al., Neuropharmacology, 36:1-11 (1997); Wu S. et al., Mol. Brain Res., 53:88-97 (1998). Cells were then cultured in DMEM supplemented with 5% dialysed foetal calf serum, 1 mM glutamine, 1 mM sodium pyruvate, 10 mM HEPES, 50 ug/ml G418 and 0.2 mg/ml hygromycin B. Confluent cultures were passaged weekly. The cells used for transfection we have referred to previously as “RGT” cells (for Rat Glutamate Transporter). Structure, expression, and functional analysis of Na+-dependent glutamate/aspartate transporter from rat brain. Proc. Natl. Acad. Sci. U.S.A. 89: 10955-10959), as a means to keep glutamate in the media to a minimum, thus preventing receptor desensitization and minimize activation by endogenously formed glutamate.

Phosphoinositide hydrolysis assays may then be performed with mGlu1a and mGlu5a receptors Schoepp D. D. et al., Neuropharmacology, 36:1-11 (1997). Transfected cells maybe seeded into 24 well culture plates at 2.5×10⁵ cells per well in medium containing no added glutamine, and cultured at 37° C. in a humidified atmosphere of 5% CO₂ in air. After 24 hours, the cells were labelled with [³H]-inositol (4 μCi/ml) for another 20 hours. Cells were washed in assay medium containing HEPES (10 mM), inositol (10 mM) and lithium chloride (10 mM). Antagonists (when tested) were added to the cell cultures 20 min prior to the addition of the agonist and then further incubated in the presence of agonist for 60 min. The reaction was terminated by replacing the medium with acetone:methanol (1:1) and the cultures incubated on ice for 20 min. Separation of the [³H]-inositol phosphates was carried out by Sep-Pak Accell Plus QMA ion exchange chromatography (Waters, Millipore Ltd., UK). The [³H]-inositol monophosphate (INS P1) fraction was eluted with 0.1M triethyl ammonium bicarbonate buffer and radioactivity was measured by liquid scintillation counting.

Cyclic-AMP (cAMP) assays may be carried out for cells expressing mGlu2,mGlu3, mGlu4, mGlu7 and mGlu8 receptors as described by Wu S. et al., Mol. Brain Research, 53:88-97 (1998). Cells may be washed with Dulbecco's phosphate buffered saline (PBS) plus 3 mM glucose and 500 mM isobutylmethylxanthine (IBMX) and preincubated for 30 min at 37° C. Each well was then washed followed by mGlu receptor agonists and/or forskolin (15 μM final concentration for mGlu2 and mGlu3, 1 μM final concentration for mGlu4, mGlu7, and mGlu8; 0.5 ml final volume per well). Cells may be incubated for 20 minutes at 37° C. and then terminated by adding 6 mM EDTA solution (0.75 ml) to each well and placing the plate in a boiling water bath. Concentrations of cAMP may be determined by an Amersham [³H]-cAMP SPA kit. Protein content in each well may be determined using the modified Bradford-Pierce assay (Pierce Chemicals, USA).

Using the above test, (+)-2-aminobicyclo[3.1.0]hexane-2,6-dicarboxylic acid (LY354740), was found to give the result shown in Table I below. Data in Table 1 is available in Schoepp D. D. et al., Neuropharmacology, 36:1-11 (1997).

TABLE 1
Summary of effects of LY354740 monohydrate on human cloned
metabotropic glutamate receptor second messenger responses.

| | | EC50 (nM) | IC50 (nM) |
|---|---|---|---|
| | | (Agonist | (Antagonist |
| Second Messenger | MGlu receptor | Activity) | Activity) |
| Decrease forskolin- | Group II clones | | |
| stimulated cAMP | human mGlu2 | 5.1 ± 0.3 | — |
| | human mGlu3 | 24.3 ± 0.5 | — |
| | Group III clones | | |
| | human mGlu4 | >100,000 | >100,000 |
| | human mGlu7 | >100,000 | >100,000 |
| | human mGlu8 | 36,000 ± 5,400 | — |
| Increase PI | Group I clones | | |
| Hydrolysis | human mGlu1 | >100,000 | >100,000 |
| | human mGlu5 | >100,000 | >100,000 |

Data are mean ± S.E.M.

Many compounds, including those discussed at length below, have such activity, and no doubt many more will be identified in the future. mGlu2/3 agonists and potentiators include, but are not limited to:
LY354740 is in clinical development as an mGlu2/3 agonist and was first taught by U.S. Pat. No. 5,750,566. Its use as an anxiolytic and psychiatric agent was disclosed in U.S. Pat. Nos. 5,882,671 and 5,661,184, respectively. Intermediates useful in preparation were first disclosed in U.S. Pat. No. 5,925,782. A process useful for preparing a Bicyclohexane derivative and intermediates was first disclosed in U.S. Pat. No. 5,726,320;
LY459477, (1S,2R,4S,5S,6S)-2-amino-4-fluorobicyclo[3.1.0]hexane-2,6-dicarboxylic acid is also disclosed in U.S. Pat. No. 5,958,960;
LY379268 and LY404039 are disclosed in U.S. Pat. No. 5,688,826. The preferred enantiomer for (1R,4S,5S,6S)-4-[(2′S)-(2′-Amino)-propionyl]amino-(2-sulfonylbicyclo[3.1.0]hexane)-4,6-dicarboxylic acid is 1R,4S,5S,6S-4-amino-2,2-dioxo-21R,4S,5S,6S -4-Amino-2,2-dioxo-2λ⁶-thia-bicyclo[3.1.0]hexane-4,6-dicarboxylic acid -thia-bicyclo[3.1.0]hexane-4,6-dicarboxyic acid;
compounds which interact with mGlu2 and/or mGlu3 to allosterically enhance receptor activity are mGlu2 receptor potentiators which include, but are not limited to, those disclosed in International Application Number PCT/US01/00643, published on Aug. 9, 2001;
furthermore, the present invention contemplates fluorinated compounds as disclosed in International Application Nos. PCT/JP99/03984, PCT/JP99/00324, and PCT/JP01/05550. See International Publication Nos. WO/0012464, WO/9938839, and WO/0200605, respectively. For example, the present invention contemplates 1S,2R,5S,6S-2-amino-6-fluoro-4-oxobicyclo[3.1.0]hexane-2,6-dicarboxylic acid; 1S,2R,4S,5S,6S-2-amino-6-fluoro-4-hydroxybicyclo[3.1.0]hexane-2,6-dicarboxylic acid; 1S,2R,3R,5S,6S-2-amino-3-fluorobicyclo[3.1.0]hexane-2,6-dicarboxylic acid; and 1S,2R,3S,5S,6S-2-amino-6-fluoro-3-hydroxybicyclo[3.1.0]hexane-2,6-dicarboxy and prodrugs, including peptidyl prodrugs, thereof;
peptidyl prodrug forms of mGlu2/3 agonists which include but are not limited to (1S,2S,5R,6S)-2-[(2′s)-(2′-amino)-propionyl]amino-bicyclo[3.1.0]hexane-2,6-dicarboxylic acid hydrochloride salt disclosed in PCT application Serial No. PCT/US01/45866, filed Dec. 21, 2001 and those disclosed in PCT/US02/00488, filed on Jan. 9, 2002. All of the patents and patent applications which have been mentioned above are used in connection with the present invention. L-alanyl prodrugs thereof are preferred;
peptidyl prodrugs forms of mGlu2/3 receptor agonists which could further encompass aspects of the instant invention are disclosed in EP Application No. 02380120.2 (U.S. Application No. 60/415936) and EP Application No. 02380121.0 (U.S. Application No. 60/415937).

It will also be understood that while the use of a single atypical antipsychotic as a first component compound is preferred, combinations of two or more atypical antipsy-chotics may be used as a first component if necessary or desired. Similarly, while the use of a single mGlu2/3 agonist or mGlu2 potentiator as a second component compound is preferred, combinations of two or more mGlu2/3 agonists may be used as a second component if necessary or desired.

While all combinations of first and second component compounds are useful and valuable, certain combinations and methods of administration are particularly valued and are preferred.

Preferred combinations which include clozapine as a first component are:
Clozapine/LY379268;
Clozapine/LY404039;
Clozapine/LY459477;
Clozapine/LY354740;
Clozapine/(1S,2S,5R,6S)-2-[(2′S)-(2′Amino)-propionyl]amino-bicyclo[3.1.0]hexane-2,6-dicarboxylic acid hydrochloride salt (oral); and
Clozapine/(1S,2R,4S,5S,6S)-4-[(2′S)-(2′-Amino)-propionyl]amino-(2-fluorobicyclo[3.1.0]hexane)-2,6-dicarboxylic acid hydrochloride.

Preferred combinations which include olanzapine as a first component are:
Olanzapine/LY379268;
Olanzapine/LY404039;
Olanzapine/LY459477;
Olanzapine/LY354740;
Olanzapine/(1S,2S,5R,6S)-2-[(2′s)-(2′-Amino)-propionyl]amino-bicyclo[3.1.0]hexane-2,6-dicarboxylic acid hydrochloride salt (oral); and
Olanzapine/(1S,2R,4S,5S,6S)-2-[(2′S)-(2′-Amino)-propionyl]amino-(4-fluorobicyclo[3.1.O]hexane)-2,6-dicarboxylic acid hydrochloride (oral).

In general, combinations and methods of treatment using clozapine or olanzapine as the first component are preferred. Furthermore, combinations and methods of treatment using LY404039 as the second component are preferred.

Furthermore, in general, it will be understood that alternative formulations to deliver components of the instant invention, particular mGlu2/3 agonists, may be accomplished via prodrugs, particularly peptidyl prodrugs.

It will be understood by the skilled reader that most or all of the compounds used in the present invention are capable of forming salts, and that the salt forms of pharmaceuticals are commonly used, often because they are more readily crystallized and purified than are the free bases. In all cases, the use of the pharmaceuticals described above as salts is contemplated in the description herein, and often is preferred, and the pharmaceutically acceptable salts of all of the compounds are included in the names of them.

Many of the compounds used in this invention are amines, and accordingly react with any of a number of inorganic and organic acids to form pharmaceutically acceptable acid addition salts. Since some of the free amines of the compounds of this invention are typically oils at room temperature, it is preferable to convert the free amines to their pharmaceutically acceptable acid addition salts for ease of handling and administration, since the latter are routinely solid at room temperature. Acids commonly employed to form such salts are inorganic acids such as hydrochloric acid, hydrobromic acid, hydroiodic acid, sulfuric acid, phosphoric acid, and the like, and organic acids, such as p-toluenesulfonic acid, methanesulfonic acid, oxalic acid, p-bromophenylsulfonic acid, carbonic acid, succinic acid, citric acid, benzoic acid, acetic acid and the like. Examples of such pharmaceutically acceptable salts thus are the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, hydroxybutyrate, glycollate, tartrate, methanesulfonate, propanesulfonate, naphthalene-1-sulfonate, naphthalene-2-sulfonate, mandelate and the like. Preferred pharmaceutically acceptable salts are those formed with hydrochloric acid, oxalic acid or fumaric acid.

Administration

The dosages of the drugs used in the present invention must, in the final analysis, be set by the physician in charge of the case, using knowledge of the drugs, the properties of the drugs in combination as determined in clinical trials, and the characteristics of the patient, including diseases other than that for which the physician is treating the patient. General outlines of the dosages, and some preferred dosages, can and will be provided here. Dosage guidelines for some of the drugs will first be given separately, in order to create a guideline for any desired combination, one would choose the guidelines for each of the component drugs.
Olanzapine: from about 0.25 to 50 mg, once/day; preferred, from 1 to 30 mg, once/day; and most preferably 1 to 25 mg once/day;
Clozapine: from about 12.5 to 900 mg daily; preferred, from about 150 to 450 mg daily;
Risperidone: from about 0.25 to 16 mg daily; preferred from about 2-8 mg daily;
Sertindole: from about 0.0001 to 1.0 mg/kg daily;
Quetiapine: from about 1.0 to 40 mg/kg given once daily or in divided doses;
Ziprasidone: from about 5 to 500 mg daily; preferred from about 50 to 100 mg daily.

In more general terms, one would create a combination of the present invention by choosing a dosage of first and second component compounds according to the spirit of the above guideline.

The adjunctive therapy of the present invention is carried out by administering a first component together with the second component in any manner which provides effective levels of the compounds in the body at the same time. All of the compounds concerned are orally available and are normally administered orally, and so oral administration of the adjunctive combination is preferred. They may be administered together, in a single dosage form, or may be administered separately.

However, oral administration is not the only route or even the only preferred route. For example, transdermal administration may be very desirable for patients who are forgetful or petulant about taking oral medicine. One of the drugs may be administered by one route, such as oral, and the others may be administered by the transdermal, percutaneous, intravenous, intramuscular, intranasal or intrarectal route, in particular circumstances. The route of administration may be varied in any way, limited by the physical properties of the drugs and the convenience of the patient and the caregiver.

The adjunctive combination may be administered as a single pharmaceutical composition, and so pharmaceutical compositions incorporating both compounds are important embodiments of the present invention. Such compositions may take any physical form which is pharmaceutically acceptable, but orally usable pharmaceutical compositions are particularly preferred. Such adjunctive pharmaceutical compositions contain an effective amount of each of the compounds, which effective amount is related to the daily dose of the compounds to be administered. Each adjunctive dosage unit may contain the daily doses of all compounds, or may contain a fraction of the daily doses, such as one-third of the doses. Alternatively, each dosage unit may contain the entire dose of one of the compounds, and a fraction of the dose of the other compounds. In such case, the patient would daily take one of the combination dosage units, and one or more units containing only the other compounds. The amounts of each drug to be contained in each dosage unit depends on the identity of the drugs chosen for the therapy, and other factors such as the indication for which the adjunctive therapy is being given.

The inert ingredients and manner of formulation of the adjunctive pharmaceutical compositions are conventional, except for the presence of the combination of the present invention. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches and suspensions. In general, compositions contain from about 0.5% to about 50% of the compounds in total, depending on the desired doses and the type of composition to be used. The amount of the compounds, however, is best defined as the effective amount, that is, the amount of each compound which provides the desired dose to the patient in need of such treatment. The activity of the adjunctive combinations do not depend on the nature of the composition, so the compositions are chosen and formulated solely for convenience and economy. Any of the combinations may be formulated in any desired form of composition. Some discussion of different compositions will be provided, followed by some typical formulations.

Capsules are prepared by mixing the compound with a suitable diluent and filling the proper amount of the mixture in capsules. The usual diluents include inert powdered substances such as starch of many different kinds, powdered cellulose, especially crystalline and microcrystalline cellulose, sugars such as fructose, mannitol and sucrose, grain flours and similar edible powders.

Tablets are prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant is necessary in a tablet formulation to prevent the tablet and punches from sticking in the die. The lubricant is chosen from such slippery solids as talc, magnesium and calcium stearate, stearic acid and hydrogenated vegetable oils.

Tablet disintegrators are substances which swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethylcellulose, for example, may be used, as well as sodium lauryl sulfate.

Enteric formulations are often used to protect an active ingredient from the strongly acid contents of the stomach. Such formulations are created by coating a solid dosage form with a film of a polymer which is insoluble in acid environments, and soluble in basic environments. Exemplary films are cellulose acetate phthalate, polyvinyl acetate phthalate, hydroxypropyl methylcellulose phthalate and hydroxypropyl methylcellulose acetate succinate. It is preferred to formulate duloxetine and duloxetine-containing combinations as enteric compositions, and even more preferred to formulate them as enteric pellets.

Tablets are often coated with sugar as a flavor and sealant. The compounds may also be formulated as chewable tablets, by using large amounts of pleasant-tasting substances such as mannitol in the formulation, as is now well-established practice. Instantly dissolving tablet-like formulations are also now frequently used to assure that the patient consumes the dosage form, and to avoid the difficulty in swallowing solid objects that bothers some patients.

When it is desired to administer the combination as a suppository, the usual bases may be used. Cocoa butter is a traditional suppository base, which may be modified by addition of waxes to raise its melting point slightly. Water-miscible suppository bases comprising, particularly, polyethylene glycols of various molecular weights are in wide use, also.

Transdermal patches have become popular recently. Typically they comprise a resinous composition in which the drugs will dissolve, or partially dissolve, which is held in contact with the skin by a film which protects the composition. Many patents have appeared in the field recently. Other, more complicated patch compositions are also in use, particularly those having a membrane pierced with innumerable pores through which the drugs are pumped by osmotic action.

### BENEFIT OF THE INVENTION

The present invention provides the advantage of treatment of psychotic conditions and mild anxiety with the atypical antipsychotics with decreased drug related side-effects typically observed with such treatment, conferring a marked and unexpected benefit on the patient. The present invention furthermore provides a potentiation of the increase in the efficacy of a first atypical antipsychotic component compound, by administration of a second component compound.

The present invention is particularly suited for use in the treatment of bipolar disorders, mania (mixed state), schizoaffective disorders characterized by the occurance of a depressive episode during the period of illness, and depression with psychotic features. Such disorders may often be resistant to treatment with an antipsychotic alone.

The present invention also is useful for the treatment of premenstrual syndrome (PMS) and anorexia nervosa. Furthermore, the present invention is useful for the treatment of the aggression/violence which may be associated with certain disorders. These disorders include, but are not limited to, mania, schizophrenia, schizoaffective disorders, substance abuse, head injury, and mental retardation.

The term “psychiatric disorder” refers to both acute and chronic psychiatric conditions, including schizophrenia, anxiety and related disorders (e.g. panic attach and stress-related cardiovascular disorders), depression (or depression in combination with psychotic episodes), bipolar disorders, psychosis, and obsessive compulsive disorders.

Psychotic conditions to be treated by the present method of adjunctive therapy include schizophrenia, schizophreniform diseases, acute mania, schizoaffective disorders, and depression with psychotic features. The titles given these conditions represent multiple disease states. The following list illustrates a number of these disease states, many of which are classified in the Diagnostic and Statistical Manual of Mental Disorders, 4^{th} Edition, published by the American Psychiatric Association (DSM). The DSM code numbers for these disease states are supplied below, when available, for the convenience of the reader.
Paranoid Type Schizophrenia 295.30
Disorganized Type Schizophrenia 295.10
Catatonic Type Schizophrenia 295.20
Undifferentiated Type Schizophrenia 295.90
Residual Type Schizophrenia 295.60
Schizophreniform Disorder 295.40
Schizoaffective Disorder 295.70
Schizoaffective Disorder of the Depressive Type
Major Depressive Disorder with Psychotic Features 296.24, 296.34

Psychoses are often associated with other diseases and conditions, or caused by such other conditions. For example, they are associated with neurological conditions, endocrine conditions, metabolic conditions, fluid or electrolyte imbalances, hepatic or renal diseases, and autoimmune disorders with central nervous system involvement. Psychoses may also be associated with use or abuse of certain substances. These substances include, but are not limited to cocaine, methylphenidate, dexmethasone, amphetamine and related substances, cannabis, hallucinogens, inhalants, opioids, phencyclidine, sedatives, hypnotics and anxiolytics. Psychotic disorders may also occur in association with withdrawal from certain substances. These substances include, but are not limited to, sedatives, hypnotics and anxiolytics. The embodiments of the present invention are useful for treatment of psychotic conditions associated with any of these conditions.

As used herein, the term “effective amount” refers to the amount or dose of the compounds, upon single or multiple dose administration to the patient, which provides the desired effect in the patient under diagnosis or treatment.

An effective amount can be readily determined by the attending diagnostician, as one skilled in the art, by the use of known techniques and by observing results obtained under analogous circumstances. In determining the effective amount or dose of compounds administered, a number of factors are considered by the attending diagnostician, including, but not limited to: the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the use of concomitant medication; and other relevant circumstances. For example, a typical daily dose may contain from about 25 mg to about 300 mg of the active ingredients. The compounds can be administered by a variety of routes, including oral, rectal, transdermal, subcutaneous, intravenous, intramuscular, bucal or intranasal routes. Alternatively, the compounds may be administered by continuous infusion.

As used herein the term “patient” refers to a mammal, such as a mouse, guinea pig, rat, dog or human. It is understood that the preferred patient is a human.

The term “treating” (or “treat”) as used herein includes its generally accepted meaning which encompasses prohibiting, preventing, restraining, and slowing, stopping, or reversing progression of a resultant symptom. As such, the methods of this invention encompass both therapeutic and prophylactic administration.

In this document, all temperatures are described in degrees Celsius, and all amounts, ratios of amounts and concentrations are described in weight units unless otherwise stated.

### EXAMPLES

The following examples are submitted for illustrative purposes only and should not be interpreted as limiting the invention in any way. A person of ordinary skill, with knowledge of this invention and of the prior art, will readily think of other subjects, other dysfunctions, and other glutamatergic substances that are readily substituted in the following examples. Also, the patents and publications cited in this disclosure refect the level of skill the art to which this invention pertains, and are herein individually incorporated by reference to the extent that they supplement, explain, provide a background for or teach methodology, techniques and/or compositions employed herein. Those of skill in the art will readily appreciate that the foregoing protocol can be used, with only minor modifications, to prepare the other compounds of the present invention.

### Example 1

Synergy between mGlu2/3 receptor agonist and atypical antipsychotic in an animal model of schizophrenia.

PCP induction of motor ambulation is a well known and widely used animal model of schizophrenia. The logic for this is based primarily on two related sets of findings:
1) PCP abuse in humans is known to provoke psychotic symptoms such as increased motor behaviors, stereotypic and cognitive disruptions; and
2) Antipsychotic drugs that are effective in the treatment of human schizophrenia are also known to attenuate stereotypic behaviors induced in rats by PCP.

Finding No. (2) indicates that PCP-induced behaviors in rates are a useful model for screening potential anti-schizophrenic drugs. Published authority for the use and reliability of this model is found in: Savitt et al., Recent Advances in the Phencyclidine Model of Schizophrenia, Am. J. Psychiatry, 148, 1301-1308 (1991); Halberstadt Al, The phencyclidine-glutamate Model of Schizophrenia, Clin. Neuropharmacol., 18, 237-249 (1995); Steinpries R. E., Behaviorial Brain Res., 74, 45-55 (1996).

In the present experiments, studies were performed in accordance with Eli Lilly and Company animal care and use policies. Male Sprague-Dawley rats (250-300 g) were group-housed (maximum of seven rats per cage) under standard laboratory conditions with ad libitum access to food and water (12 h light/dark cycle), for at least 1 day before use.

Activity Assessment compounds were tested against PCP-induced motor activation (ambulations) in rats. Behavioral parameters were monitored in transparent, shoe-box cages that measured 45×25×20 cm, with a 1 cm depth of wood chips on the cage floor and a metal grill on top of the cage. Rectangular photocell monitors (Hamilton Kinder, Poway, Calif.) with a bank of 12 photocell beams (8×4 formation) surrounded each test cage. A lower rack of photocell beams was positioned 5 cm above the cage floor to enable detection of the location of the animals body, while an upper bank positioned 10 cm above the first tabulated rearing activity. Ambulations (locomotor activity) and rearing were recorded by the computer and stored for each test session as discussed in Male Sprague-Dawley rats were generally food-fasted 12-18 hours prior to the experiment. In some experiments, rats were allowed food and water ad libitum prior to the experiment. On the test day animals were placed in the test cage for a 30 min habituation period before to testing to allow for acclimation to the test cage environment. Following this habituation period, animals were administered challenges of phencyclidine (PCP) (5 mg/kg s.c.) or 0.9% NaCl vehicle (1 ml/kg) and behavioral assessment began immediately following their administration. Animals were monitored over a 60 min period in all instances. Test drugs or vehicle were administered at various pretreatment times prior to the PCP challenge. Cartmell J., J. Pharmacol. Exp. Ther. 291: 161-170 (1999) and Cartmell J., Naunyn-Schmiedebeig's Archives Pharmacology 361: 39-46 (2000).

Statistical analysis. Statistical Analysis were carried out using the GraphPad PRISM statistical/graphing package (GraphPad, SanDiego, Calif.). Data were analyzed using a one-way analysis of variance (ANOVA) and post-hoc comparisons were performed using Dunnett's multiple comparisons test.

Materials. PCP was obtained from Sigma (St. Louis, Mo.). Clozapine was purchased from Research Biochemicals International (Natick, Mass.). MGlu2/3 receptor agonists were synthesized as described U.S. Pat. No. 5,958,960 (LY459477) and U.S. Pat. No. 5,688,826 (LY404039).

The rats were tested 30 minutes post-injection of the test compound or vehicle. Behaviors were monitored over a 60 minute time period following S.C. injection of PCP or vehicle. Data (mean±S.E.) are presented as the total number of behaviors expressed during the timer period. P<0.05, when compared to the corresponding vehicle.

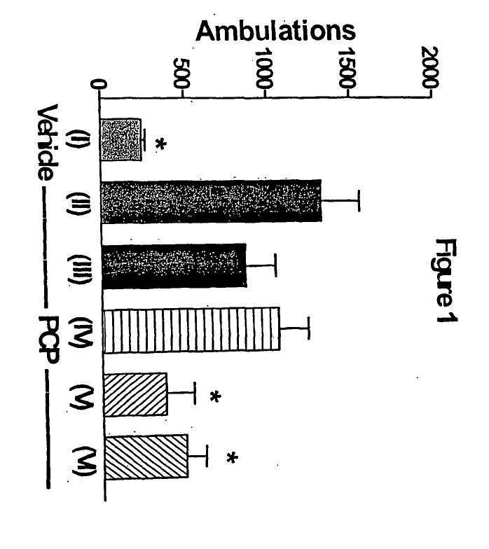

As shown in FIG. 1, there was a large induction of motor ambulations by 5 mg/kg PCP (S.C.) (II), compared to the vehicle (I). Clozapine (3 mg/kg) had a relatively small impact on PCP-induced ambulations (III), while the selected mGlu2/3 receptor agonist, LY404039 at 1 mg/kg had statistically insignificant effect on PCP-induced ambulations (IV). However, together clozapine (3 mg/kg) and LY404039 (1 mg/kg) (V) produced a synergistic interaction, reducing PCP-induced ambulations to a level even less than that of clozapine alone at 10 mg/kg (VI).

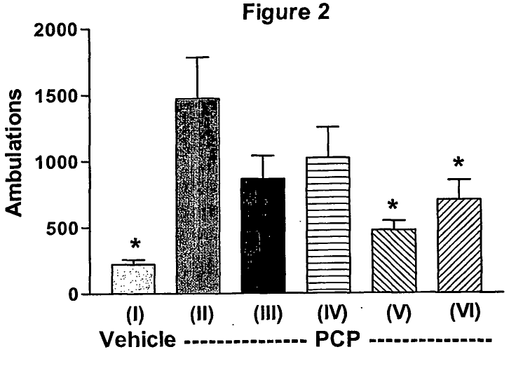

Further, as shown in FIG. 2, there was a large induction of motor ambulations by 5 mg/kg PCP (S.C.) (II), compared to the vehicle (I). Clozapine (3 mg/kg) had a relatively small impact on PCP-induced ambulations (III), while the selected mGlu2/3 receptor agonist LY379268 at 1 mg/kg had a smaller and statistically insignificant effect on PCP-indused ambulations (IV). However, together clozapine (3 mg/kg) and LY379268 (1 mg/kg)(V) produced a synergistic interaction, reducing PCP-induced ambulations to a level even less than that of clozapine alone at 10 mg/kg (VI).

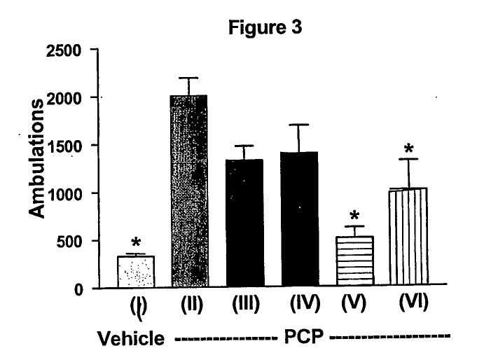

FIG. 3 shows there was a large induction of motor ambulations by 5 mg/kg PCP (S.C.) (II), compared to the vehicle (1). The selected mGlu2/3 receptor agonist, LY459477, at 1 mg/kg had a relatively small impact on PCP-induced ambulations (III), while clozapine (1 mg/kg) had a statistically insignificant effect on PCP-induced ambulations (IV). However, together clozapine (1 mg/kg) and LY459477 (1 mg/kg)(V) produced a synergistic interaction, reducing PCP-induced ambulations to a level even less than that of clozapine alone at 10 mg/kg (VI).

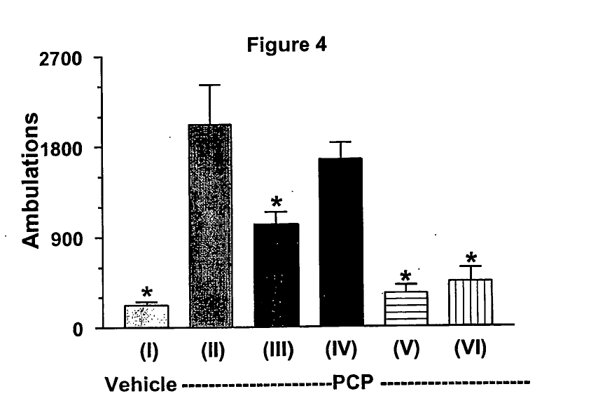

FIG. 4 shows there was a large induction of motor ambulations by 5 mg/kg PCP (S.C.) (II), compared to the vehicle (I). Clozapine (3 mg/kg) had a significant impact on PCP-induced ambulations (m), while the selected mGlu2/3 receptor agonist, LY354740, at 10 mg/kg had a statistically insignificant effect on PCP-induced ambulations (IV). However, together clozapine (3 mg/kg) and LY354740 (10 mg/kg)(V) produced a synergistic interaction, reducing PCP-induced ambulations to a level even less than that of clozapine alone at 10 mg/kg (VI).

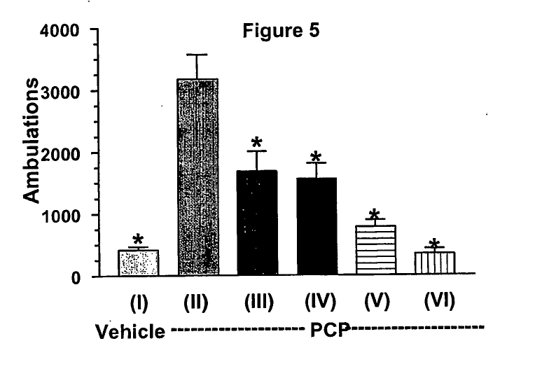

As shown in FIG. 5, there was a large induction of motor ambulations by 5 mg/kg PCP (S.C.) (II), compared to the vehicle (I). Olanzapine (1 mg/kg) had a significant impact on PCP-induced ambulations (II), and the selected mGlu2/3 receptor agonist, LY404039 at 1 mg/kg had statistically significant effect on PCP-induced ambulations (IV) as well. Together olanzapine (1 mg/kg) and LY 404039 (1 mg/kg) (V) produced a synergistic interaction, reducing PCP-induced ambulations to a level comparable that of olanzapine alone at 3 mg/kg (II).

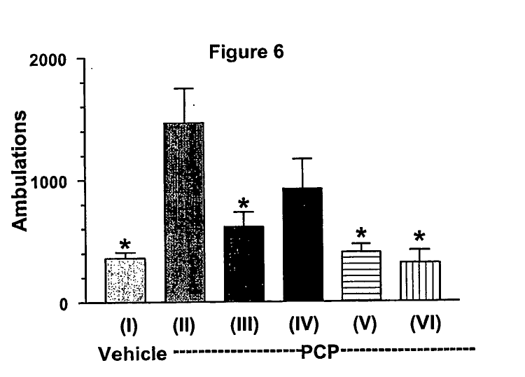

As shown in FIG. 6, there was a large induction of motor ambulations by 5 mg/kg PCP (S.C.) (II), compared to the vehicle (I). Olanzapine (1 mg/kg) had a significant impact on PCP-induced ambulations (II), and the selected mGlu2/3 receptor agonist, LY379268 at 1 mg/kg had a statistically insignificant effect on PCP-induced ambulations (IV) as well. Together olanzapine (1 mg/kg) and LY379268 (1 mg/kg) (V) produced a synergistic interaction, reducing PCP-induced ambulations to a level comparable that of olanzapine alone at 3 mg/kg (VI).

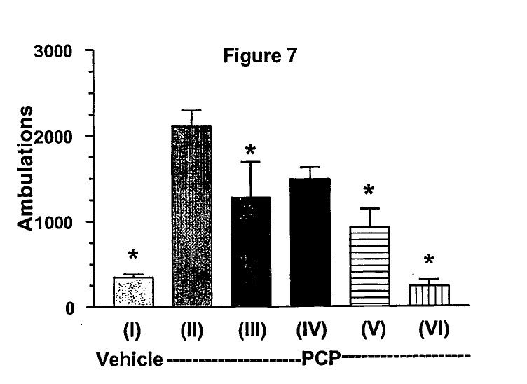

As shown in FIG. 7, shows a large induction of motor ambulations by 5 mg/kg PCP (S.C.) (II), compared to the vehicle (I). Olanzapine (1 mg/kg) had a significant impact on PCP-induced ambulations (E), and the selected mGlu2/3 receptor agonist, LY459477 at 1 mg/kg had a statistically insignificant effect on PCP-induced ambulations (IV) as well. Together olanzapine (1 mg/kg) and LY459477 mGlu2/3 receptor agonist (1 mg/kg) (V) produced a synergistic interaction, reducing PCP-induced ambulations to a level comparable that of olanzapine alone at 3 mg/kg (VI).

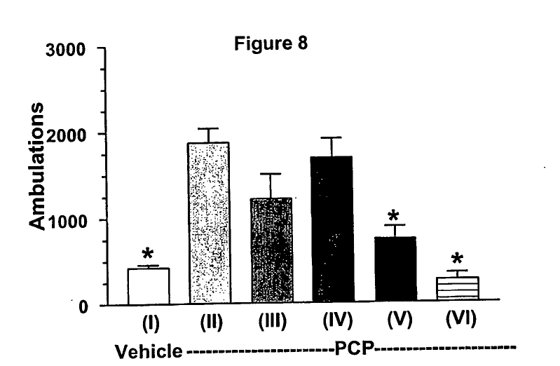

As shown in FIG. 8, there was a large induction of motor ambulations by 5 mg/kg PCP (S.C.) (II), compared to the vehicle (I). Olanzapine (1 mg/kg) had minimal impact on PCP-induced ambulations (II), and the selected mGlu2/3 receptor agonist, LY354740 at 10 mg/kg had a statistically insignificant effect on PCP-induced ambulations (IV) as well. Together olanzapine (1 mg/kg) and LY354740 mGlu2/3 receptor agonist (10 mg/kg) (V) produced a synergistic interaction, reducing PCP-induced ambulations to a level comparable that of olanzapine alone at 3 mg/kg (VI).

The present invention therefore provides an improved method of treatment of psychosis via decreasing the side effects of an atypical antipsychotic at efficacious doses.

### Example 2

A first step in treating humans is generally determining that a particular patient exhibits the symptoms of a psychotic behaviour such as Schizophrenia or Schizophreniform Disorder or Schizoaffective Disorder or Delusional Disorder or Brief Psychotic Disorder or Psychotic Disorder Due to a General Medical Condition or Psychotic Disorder Not Otherwise Specified. This determination is made by a person skilled in the art using a number of readily available diagnostic procedures. In general, the presence of typical DSMIV psychotic dysfunctions in humans can be ascertained via observation, diagnosis, family history, questionnaires or interviews. The success of treatment is measured by monitoring and recording the abatement of the symptoms of the treated behavioral disorder.

In addition, the present invention provides for kits with unit doses of mGlu2/3 receptor agonists and an atypical antipsychotic either in oral or injectable doses. In addition to the containers containing the unit doses will be a informational package insert describing the use and attendant benefits of the drugs in treating psychiatric disorders. Preferred combinations and unit doses include those described herein above.

1. A pharmaceutical composition which comprises a first component which is an atypical antipsychotic and a second component which is mGlu2/3 receptor agonist.

2. A method for treating a patient suffering from or susceptible to a psychiatric disorder, comprising administering to said patient an amount of a first component which is an atypical antipsychotic, in combination with an amount of a second component which is a mGlu2/3 receptor agonist.

3. A pharmaceutical composition which comprises a first component which is clozapine and a second component which is (1R,4S,5S,6S)-4-[(2′S)-(2′-Amino)-propionyl]amino-(2-sulfonylbicyclo[3.1.0]hexane)-4,6-dicarboxylic acid.

4. A method for treating a patient suffering from or susceptible to a psychiatric disorder, comprising administering to said patient an amount of a first component which is clozapine, in combination with an amount of a second component which is (1R,4S,5S,6S)-4-[(2'S)-(2′-Amino)-propionyl]amino-(2-sulfonylbicyclo[3.1.0]hexane)-4,6-dicarboxylic acid.

5. A pharmaceutical composition which comprises a first component which is clozapine and a second component which is (1R,4R,5S,6R)-4-amino-(2-oxabicyclo[3.1.0]hexane)-4,6-dicarboxylic acid.

6. A method for treating a patient suffering from or susceptible to a psychiatric disorder, comprising administering to said patient an amount of a first component which is clozapine, in combination with an amount of a second component which is (1R,4R,6S,6R)-4-amino-(2-oxabicyclo[3.1.0]hexane)-4,6-dicarboxylic acid.

7. A pharmaceutical composition which comprises a first component which is clozapine and a second component which is (1S,2R,4S,5S,6S)-2-amino-4-fluorobicyclo[3.1.0]hexane-2,6-dicarboxylic acid.

8. A method for treating a patient suffering from or susceptible to a psychiatric disorder, comprising administering to said patient an amount of a first component which is clozapine in combination with an amount of a second component which is (1S,2R,4S,5S,6S)-2-amino-4-fluorobicyclo[3.1.0]hexane-2,6-dicarboxylic acid.

9. A pharmaceutical composition which comprises a first component which is clozapine and a second component which is (+)-2-aminobicyclo[3.1.0]hexane-2,6-dicarboxylic acid.

10. A method for treating a patient suffering from or susceptible to a psychiatric disorder, comprising administering to said patient an amount of a first component which is clozapine in combination with an amount of a second component which is (+)-2-aminobicyclo[3.1.0]hexane-2,6-dicarboxylic acid.

11. A pharmaceutical composition which comprises a first component which is olanzapine and a second component which is (1R,4S,5S,6S)-4-[(2′S)-(2′-Amino)-propionyl]amino-(2-sulfonylbicyclo[3.1.0]hexane)-4,6-dicarboxylic acid.

12. A method for treating a patient suffering from or susceptible to a psychiatric disorder, comprising administering to said patient an amount of a first component which is olanzapine in combination with an amount of a second component which is (1R,4S,5S,6S)-4-[(2+S)-(2′-Amino)-propionyl]amino-(2-sulfonylbicyclo[3.1.0]hexane)-4,6-dicarboxylic acid.

13. A pharmaceutical composition which comprises a first component which is olanzapine and a second component which is (1R,4R,5S,6R)-4-amino-(2-oxabicyclo[3.1.0]hexane)-4,6-dicarboxylic acid.

14. A method for treating a patient suffering from a susceptible to a psychiatric disorder, comprising administering to said patient an amount of a first component which is olanzapine in combination with an amount of a second component which is (1R,4R,5S,6R)-4-amino-(2-oxabicyclo[3.1.0]hexane)-4,6-dicarboxylic acid.

15. A pharmaceutical composition which comprises a first component which is olanzapine and a second component which is (1S,2R,4S,6S)-2-amino-4-fluorobicyclo[3.1.0]hexane -2,6-dicarboxylic acid.

16. A method for treating a patient suffering from or susceptible to a psychiatric disorder, comprising administering to said patient an amount of a first component which is olanzapine in combination with an amount of a second component which is (1S,2R,4S,6S)-2-amino-4-fluorobicyclo[3.1.0]hexane -2,6-dicarboxylic acid.

17. A pharmaceutical composition which comprises a first component which is olazapine and a second component which is (+)-2-aminobicyclo[3.1.0]hexane -2,6-dicarboxylic acid.

18. A method for treating a patient suffering from or susceptible to a psychiatric disorder, comprising administering to said patient an amount of a first component which is olanzapine in combination with an amount of a second component which is (+)-2-aminobicyclo[3.1.0]hexane -2,6- dicarboxylic acid.

19. A pharmaceutical composition which comprises a first component which is an atypical antipsychotic and a second component which is a compound which allosterically enhances receptor activity for mGlu2 or mGlu3.

20. A method for treating a patient suffering from or susceptible to a psychiatric disorder, comprising administering to said patient an amount of a first component which is an atypical antipsychotic in combination with an amount of a second component which is a compound which allosterically enhances receptor activity for mGlu2 or mGlu3.

21. A pharmaceutical composition which comprises a first component which is an atypical antipsychotic and a second component which is a compound which allosterically enhances receptor activity for mGlu2 and mGlu3.

22. A method for treating a patient suffering from or susceptible to a psychiatric disorder, comprising administering to said patient an amount of a first component which is an atypical antipsychotic in combination with an amount of a second component which is a compound which allosterically enhances receptor activity for mGlu2 and mGlu3.
